(19) United States
(12) Patent Application Publication
(10) Pub. No.: US 2017/0196639 A1
(43) Pub. Date: Jul. 13, 2017

(54) DUAL BAND POWER AMPLIFIER CIRCUIT FOR MICROWAVE ABLATION

(71) Applicant: COVIDIEN LP, MANSFIELD, MA (US)

(72) Inventor: ZHONGYU LIAO, SHANGHAI (CN)

(73) Assignee: COVIDIEN LP, MANSFIELD, MA (US)

(21) Appl. No.: 15/324,287

(22) PCT Filed: Jul. 14, 2014
(86) PCT No.: PCT/CN2014/082156

§ 371 Date: Jan. 6, 2017

(51) Int. Cl. A61B 18/18; H03F 3/21; H03F 1/56

(52) U.S. Cl. CPC A61B 18/1815; H03F 1/56; H03F 3/21; H03F 2200/255; H03F 2200/111; H03F 2200/423; A61B 2018/00577

(57) ABSTRACT

A dual band power amplifier includes a power amplifier, a first matching circuit, a first auxiliary circuit, a second matching circuit, and a second auxiliary circuit. The power amplifier has inputs and outputs, and configured to amplify input signals at a first and second frequency. The first matching circuit electrically connected to the output of the power amplifier and configured to match a load impedance to an output impedance at the first frequency. The first matching circuit and the first auxiliary circuit configured to match the load impedance to the output impedance at the second frequency. The second matching circuit electrically connected to the input of the power amplifier and configured to match a source impedance to an input impedance at the first frequency. The second matching circuit and the second auxiliary circuit configured to match the source impedance to the input impedance at the second frequency.

### CROSS REFERENCE TO RELATED APPLICATION

The present application is a U.S. National Stage Application under 35 U.S.C. §371(a) of PCT/CN2014/082156 filed Jul. 14, 2014, the entire contents of which are incorporated by reference herein.

### BACKGROUND

Technical Field

The present disclosure relates to a dual band power amplifier circuitry for a microwave generator. More particularly, the present disclosure relates to a microwave ablation generator that includes one dual band power amplifier circuitry which concurrently amplifies signals having one frequency and signals having another frequency.

Background of Related Art

Generally, a microwave source of a microwave generator generates a signal having a single frequency whose power is amplified to a certain magnitude by a power amplifier. The generated signal is travelling from the microwave source to the power amplifier through a transmission line. Each end of the transmission line is a critical boundary point where the generated signal might be reflected when impedances around the critical boundary point do not match. The travelling signal and the reflected signals may overlap or superimpose to each other, meaning that these signals are additive at some points and are subtractive at other points.

The travelling signal and the reflected signals may become a standing wave, which causes power waste in the microwave generator resulting in poor efficiencies and resulting in unsatisfactory performance. Furthermore, when the travelling signal and the reflected signals are added, summed up power at one place along the transmission line or at one electrical component, the added power may go over the capacity of the circuits in the microwave generator and cause destruction of the circuits. For this reason, an impedance matching circuit is placed between the microwave source and the transmission line or between the transmission line and the power amplifier. Or, generally, an impedance matching circuit is placed between the microwave source and the input of the power amplifier and another impedance matching circuit is placed between the output of the amplifier and a load of the microwave generator (e.g., an energy delivery device).

In ablation procedures, a signal having a different frequency may be needed to treat different types of tissue. Thus, microwave generators need to generate signals having different frequencies to meet the needs for ablating different types of tissues. The impedance matching circuit for one frequency may mismatch the impedance for another frequency. In other words, when the microwave source switches to generate signals having a different frequency, the impedance matching circuit, which has matched the impedance at the previous frequency, cannot match the impedance at the current frequency. Thus, the microwave generator has to have a number of impedance matching circuits as same as the number of frequencies that the microwave generator is capable of generating. That increases the size of and the cost of making the microwave generator.

### SUMMARY

The present disclosure features a dual band amplifier circuitry for a microwave generator, which include impedance matching circuits that concurrently match impedance for two different and independent frequencies.

In an embodiment, the dual band power amplifier includes a power amplifier, a first matching circuit, a first auxiliary circuit, a second matching circuit, and a second auxiliary circuit. The power amplifier has an input and an output, and is configured to amplify input signals at a first frequency and a second frequency. The first matching circuit is electrically connected to the output of the power amplifier and is configured to match a load impedance to an output impedance of the power amplifier at the first frequency. The first auxiliary circuit is electrically connected to the output of the power amplifier and has at least two shunt stubs. The first matching circuit and the first auxiliary circuit are configured to match the load impedance to the output impedance of the power amplifier at the second frequency. The second matching circuit is electrically connected to the input of the power amplifier and configured to match a source impedance to an input impedance of the power amplifier at the first frequency. The second auxiliary circuit is electrically connected to the input of the power amplifier and has at least two shunt stubs. The second matching circuit and the second auxiliary circuit are configured to match the source impedance to the input impedance of the power amplifier at the second frequency. The at least two shunt stubs of the first auxiliary circuit and the at least two shunt stubs of the second auxiliary circuit have a length substantially equal to a quarter wavelength of the first frequency.

In another embodiment, the frequency is substantially greater than the second frequency. The first frequency may be 2,450 megahertz (MHz) and the second frequency may be 915 MHz.

In another embodiment, the first matching circuit, the second matching circuit, the first auxiliary circuit, the second auxiliary circuit, and the power amplifier are electrically connected by a transmission line. The characteristic impedance of an input portion of the transmission line is equal to the source impedance. The characteristic impedance of an output portion of the transmission line is equal to the load impedance.

In another embodiment, at the first frequency, an imaginary part of the source impedance and an imaginary part of the input impedance of the power amplifier have a substantially equal magnitude and are 180° out of phase and, at the first frequency, an imaginary part of the load impedance and an imaginary part of the output impedance of the power amplifier have a substantially equal magnitude and are 180° out of phase.

In another embodiment, at the second frequency, an imaginary part of the source impedance and an imaginary part of the input impedance of the power amplifier have a substantially equal magnitude and are 180° out of phase and, at the second frequency, an imaginary part of the load impedance and an imaginary part of the output impedance of the power amplifier have a substantially equal magnitude and are 180° out of phase.

In another embodiment, the at least two shunt stubs of the first auxiliary circuit are electrically connected in parallel to the output of the power amplifier.

In another embodiment, the at least two shunt stubs of the second matching circuit are electrically connected in parallel to the input of the power amplifier.

In yet another embodiment, a microwave generator includes a source of microwave energy, a power amplifier, a first matching circuit, a first auxiliary circuit, a second matching circuit, and a second auxiliary circuit. The source of microwave energy generates input signals at a first frequency and a second frequency, and has a source impedance. The power amplifier has an input and an output and is configured to amplify the input signals at the first frequency and the second frequency. The first matching circuit is electrically connected to the output of the power amplifier and is configured to match a load impedance to an output impedance of the power amplifier at the first frequency. The first auxiliary circuit is electrically connected to the output of the power amplifier and has at least two shunt stubs. The first matching circuit and the first auxiliary circuit are configured to match the load impedance to the output impedance of the power amplifier at the second frequency. The second matching circuit is electrically connected to the input of the power amplifier and configured to match the source impedance to an input impedance of the power amplifier at the first frequency. The second auxiliary circuit is electrically connected to the input of the power amplifier and has at least two shunt stubs. The second matching circuit and the second auxiliary circuit are configured to match the source impedance to the input impedance of the power amplifier at the second frequency. The at least two shunt stubs of the first auxiliary circuit and the at least two shunt stubs of the second auxiliary circuit have a length substantially equal to a quarter wavelength of the first frequency.

In another embodiment, the frequency is substantially greater than the second frequency. The first frequency may be 2,450 megahertz (MHz) and the second frequency may be 915 MHz.

In another embodiment, the first matching circuit, the second matching circuit, the first auxiliary circuit, the second auxiliary circuit, and the power amplifier are electrically connected by a transmission line. The characteristic impedance of an input portion of the transmission line is equal to the source impedance. The characteristic impedance of an output portion of the transmission line is equal to the load impedance.

In another embodiment, at the first frequency, an imaginary part of the source impedance and an imaginary part of the input impedance of the power amplifier have a substantially equal magnitude and are 180° out of phase and, at the first frequency, an imaginary part of the load impedance and an imaginary part of the output impedance of the power amplifier have a substantially equal magnitude and are 180° out of phase.

In another embodiment, at the second frequency, an imaginary part of the source impedance and an imaginary part of the input impedance of the power amplifier have a substantially equal magnitude and are 180° out of phase and, at the second frequency, an imaginary part of the load impedance and an imaginary part of the output impedance of the power amplifier have a substantially equal magnitude and are 180° out of phase.

In another embodiment, the at least two shunt stubs of the first auxiliary circuit are electrically connected in parallel to the output of the power amplifier.

In yet another embodiment, the at least two shunt stubs of the second matching circuit are electrically connected in parallel to the input of the power amplifier.

### BRIEF DESCRIPTION OF THE DRAWINGS

The present disclosure may be understood by reference to the accompanying drawings, when considered in conjunction with the subsequent, detailed description, in which:

FIG. 1 is an illustration of a microwave ablation system in accordance with embodiments of the present disclosure;

FIG. 2 is a functional block diagram of a microwave generator of the microwave ablation system of FIG. 1 in accordance with embodiments of the present disclosure;

FIG. 3 is a functional circuit diagram of a general microwave generator;

FIG. 4 is a circuit diagram of a dual band power amplifier circuitry in accordance with embodiments of the present disclosure; and

FIG. 5 is a flowchart illustrating a process for designing a dual band power amplifier circuitry in accordance with embodiments of the present disclosure.

### DETAILED DESCRIPTION

A dual power amplifier circuitry according to the present disclosure enables a dual band microwave generator to achieve good performance concurrently at two different and independent frequencies. In other words, the dual power amplifier circuitry concurrently matches impedance at two different and independent frequencies. The dual band microwave generator includes a microwave source that generates a signal having a microwave range frequency. The microwave source inherently has an impedance value, the source impedance. An energy delivery device is connected to an output port of the microwave generator so that the generated signal is transmitted to the energy delivery device. This energy delivery device also inherently has an impedance value, the load impedance. Between the microwave source and the energy delivery device or between the source impedance and the load impedance, the dual band power amplifier circuitry is placed and amplifies the generated signal to a magnitude sufficient for intended performance at two independent and different microwave frequencies. Due to mismatches between the source impedance and the input impedance of the power amplifier, and between the output impedance of the power amplifier and the load impedance, the dual power amplifier circuitry includes input and output impedance matching circuits that match the source impedance to the input impedance of the power amplifier and the load impedance to the output impedance of the power amplifier at two independent and different microwave frequencies.

The term “microwave frequency” generally refers to electromagnetic waves in the frequency range of 300 megahertz (MHz) (3×10⁸ cycles per second) to 300 gigahertz (GHz) (3×10¹¹ cycles per second).

Transmission lines connect among the microwave source, the dual power amplifier circuitry, and the energy delivery device. The term transmission lines generally refer to any transmission medium that can be used for propagation of signals from one component to another.

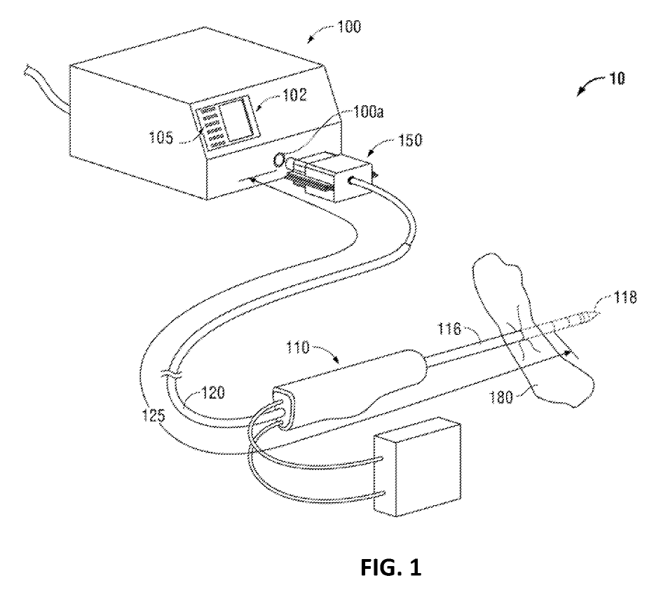

FIG. 1 illustrates a microwave generator system 10 which includes a microwave generator 100, an energy delivery device 110, a coaxial transmission cable 120, and an isolation apparatus 150. The microwave generator 100 generates a signal having a microwave range frequency, which is suitable for intended purposes, such as ablating tissues. The microwave generator 100 includes an output port 100a, a display 102, and buttons 105. The microwave generator 100 outputs the generated signal to the output port 100a, which is connectable with a coaxial cable. The display 102 may display values for setting items, such as frequency and power of the generated signal, and graphical information of the generated signal, such as types (e.g., saw tooth type, sinusoidal type, a square wave type, etc.). The buttons 105 may be used to set the setting items so that a user may set a frequency, an amount of power, and a type of the signal to be generated.

The microwave energy delivery device 110 includes a handle 116, an antenna 118, and coaxial transmission cable 120. The handle 116 is a transmission portion of the energy delivery device 110 proximal to the antenna 118, which radiates the delivered energy into the environment. The coaxial transmission cable 120 is the transmission path of the generated signal from the output port 100a to the antenna 118. Transmission path 125 includes the isolation apparatus 150, the coaxial transmission cable 120, and the handle 116. The microwave energy delivery device 110 may include a percutaneous device having a sharpened tip configured to penetrate tissue to ablate target tissue.

The isolation apparatus 150 is connected between the microwave generator 100 and the microwave energy delivery device 110 through a coaxial cable 100a of the microwave generator 100. The isolation apparatus 150 may also be used with a catheter insertable microwave energy delivery device, a skin surface treatment microwave energy delivery device and a deployable microwave energy delivery device or other suitable device configured to deliver microwave energy to tissue 180. The isolation apparatus 150 together with the energy delivery device 110 inherently have an impedance value, which is called the load impedance. The load impedance affects efficiency of power transmission from the microwave generator 100 to the energy delivery device 110. For explaining purposes, the load impedance is considered to be equal to 50 ohms.

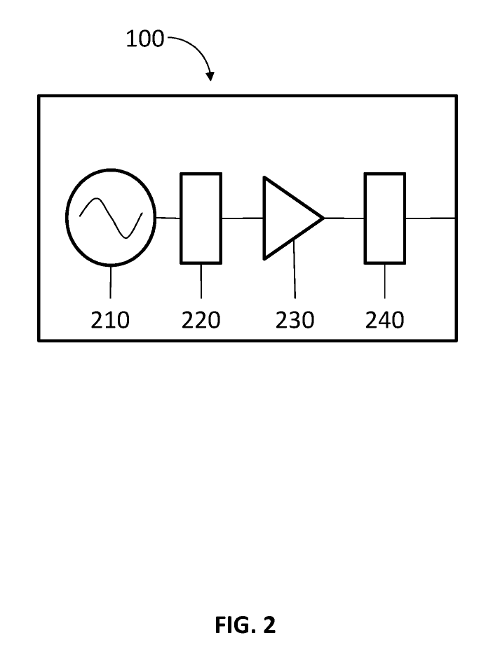

FIG. 2 shows a functional block diagram illustrating the microwave generator 100 of the microwave generator system 10 of FIG. 1. The microwave generator 100 includes a microwave source 210, an input impedance matching circuitry 220, a power amplifier 230, and the output impedance matching circuitry 240. The microwave source 210 generates a signal having a specific frequency suitable for its intended performance, such as ablating target tissue. This specific frequency may be selected by considering effective heating and heating boundary according to the type of target tissue. For example, the microwave source 210 generates a signal having 915 megahertz (MHz) to ablate liver tissue or a signal having 2,450 MHz to ablate liver or lung tissue. In one aspect of the present disclosure, the microwave source 210 may generate signals having a different frequency suitable for ablating other types of tissue, such as malignant cells in stomach, liver, lung, kidney, intestine, etc. and such frequencies may be 150, 433, 915, 2,450, and 5,800 MHz. This list of frequencies is provided as an example and does not limit the scope of frequency other than the microwave frequency range.

Inherently, the microwave source 210 has an impedance value, which is called the source impedance. The input impedance of the power amplifier 230 together with the source impedance affects efficiencies of power transfer between the microwave source 210 and the power amplifier 230. For explaining purposes, the microwave source 210 is considered to have an impedance value of 50 ohms.

When a signal is generated by the microwave source 210, the generated signal may not have sufficient power to perform intended operations. The power amplifier 230 then amplifies the generated signal to a certain magnitude sufficient for the intended operations. Here, the power amplifier 230 also inherently has an impedance value. In one aspect of the present disclosure, the power amplifier 230 may have two different impedance values: one is an input impedance value of the power amplifier 230 seen from the side of the microwave source 210 and the other one is an output impedance value of the power amplifier 230 seen from the output of the microwave generator 100. The former is named the input impedance of the power amplifier and the latter is named the output impedance of the power amplifier for explanatory purposes in this disclosure.

The microwave source 210, the power amplifier 230, and the output port of the microwave generator 100 are generally connected by transmission lines. If the impedance of the transmission line is not equal to or does not match the source impedance of the microwave source 210, the generated signal is reflected at the connection point from the transmission line to the microwave source 210. Also, when the impedance of the transmission line and the input impedance of the power amplifier 230 do not match, the signal travelling through the transmission line is also reflected at the connection point from the power amplifier 230 to the transmission line. These reflected signals and the originally generated signal are superimposed to each other and may generate a standing wave, which consumes power and may be destructive of the circuits of the microwave generator 100 where the reflected signals are added to the generated signal and the magnitude resulting from superposition potentially exceeds the capacity of some circuits in the microwave generator 100. The input impedance matching circuitry 220 matches the source impedance of the microwave source 210 to the input impedance of the power amplifier 230 to prevent such unintended damages.

The input impedance matching circuitry 220 may be capable of concurrently matches the input impedance of the power amplifier 230 to the source impedance of the microwave source 210 at two independent and different frequencies. As described above, the source impedance of the microwave source 210 is typically 50 ohms. Similarly, the impedance of the transmission line that connects each component of the microwave generator 100 is also typically 50 ohm. The characteristic impedance of the transmission line is measured along the transmission line by dividing voltage signal by current signal. Thus, signals are not reflected along the transmission line from the microwave source and to the power amplifier when the characteristic impedance of the transmission line is constant, because the source impedance and load impedance are matched to the characteristic impedance of the transmission line, i.e., 50 ohms.

In the same way, the output impedance matching circuitry 240 matches the load impedance, which is an impedance value of an energy delivery device connected to the microwave generator 100, such as the energy delivery device 110 of FIG. 1, to the output impedance of the power amplifier 230. In this way, the microwave generator 100 is able to transmit the generated signal via the power amplifier 230 to the energy delivery device efficiently with little or no reflection or energy loss.

Generally, impedance is in a form of complex number, i.e., a+jb, where a and b are real numbers and j represents an imaginary part of the complex number. For example, if the input impedance of the power amplifier 230 is a+jb, the input impedance matching circuit matches the source impedance to the conjugate of the input impedance of the power amplifier 230, i.e., a−jb. In the same way, when the output impedance of the power amplifier 230 is c+jd, the output impedance matching circuit 240 matches the load impedance to the conjugate of the output impedance of the power amplifier 230, i.e., c−jd.

However, when the microwave source 210 generates a signal matching circuits 220 and 240 will typically only be able to match impedances at a single frequency, f₁. This limitation is primarily a function of the components of the matching circuits, specifically capacitors and inductors. For example, a capacitor, which has a capacitance value of C, has an

$$\frac{1}{j ω C} = \frac{2 π}{jfC} ,$$

impedance value of where w is an angular frequency of the frequency f and j represents the imaginary part of a complex value, and an inductor, which has an inductance value

$$j ω L = \frac{j fL}{2 π} .$$

of L, has an impedance value of Thus, the impedances of capacitors and inductors depend on a frequency of the signal.

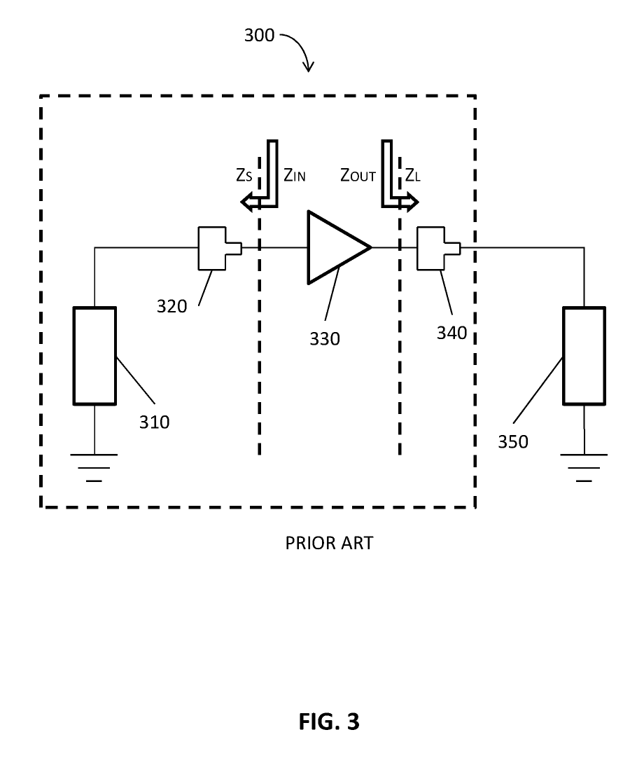

To further describe the impedance matching, reference is made to FIG. 3. In FIG. 3 a microwave generator 300 is a typical exemplary microwave generator, in which an impedance matching circuit can match impedance only at a single frequency. The microwave generator 300 includes microwave source 310, an input impedance matching circuit 320, a power amplifier 330, and an output impedance matching circuit 340. An energy delivery device 350 is connected to the output impedance matching circuit 340 or an output port of the microwave generator 300. The source impedance Z_{S} represents the impedance of a microwave source 310, which generates a signal at frequency f₁, and the load impedance Z_{L} represents an impedance of the energy delivery device 350.

Source impedance Z_{S} may represent an impedance value of the microwave source 310 or an overall impedance value of all electrical components on the input side of the power amplifier 330, which includes the input matching circuit 320, the microwave source 310, and transmission lines. In other words, the source impedance Z_{S} is an impedance value seen from the input port side of the power amplifier 330. Z_{EN} is the input impedance of the power amplifier 330. The load impedance Z_{L} represents an impedance value of the energy delivery device 350 or an overall impedance value of all electrical components in the output side of the power amplifier 330, which includes the output matching circuit 340, the energy delivery device 350, and the transmission lines. In other words, the load impedance Z_{L} is an impedance value seen from the output port side of the power amplifier 330. Z_{OUT} is the output impedance of the power amplifier 330.

The input impedance Z_{EN} of the power amplifier 330 is not generally equal to the source impedance Z_{S} and the output impedance Z_{OUT} of the power amplifier 330 is not generally equal to the load impedance Z_{L} and thus require the use of matching circuits as described above. The input impedance matching circuit 320 matches the source impedance Z_{S} to the input impedance Z_{EN} of the power amplifier 330. Thus, there may be no signal reflection between the power amplifier 330 and the source 310. In the same way, the output impedance matching circuit 340 matches the load impedance Z_{L} to the output impedance Z_{OUT} of the power amplifier. Thus, there may be no signal reflection between the power amplifier 330 and the load 350.

Nevertheless, since the input and output impedance matching circuits 320 and 340 are designed to match impedance at a single frequency f₁ of the signal generated by the microwave generator 300, the input and output impedance matching circuits 320 and 340 may not be capable of matching impedance at another frequency f₂, which is different from frequency f₁. As described above, the reactive and/or capacitive components in the input and output impedance matching circuits 320 and 340 change the source and load impedances at frequency f₂, resulting mismatch between the source impedance and the input impedance of the power amplifier and between the load impedance and the output impedance of the power amplifier.

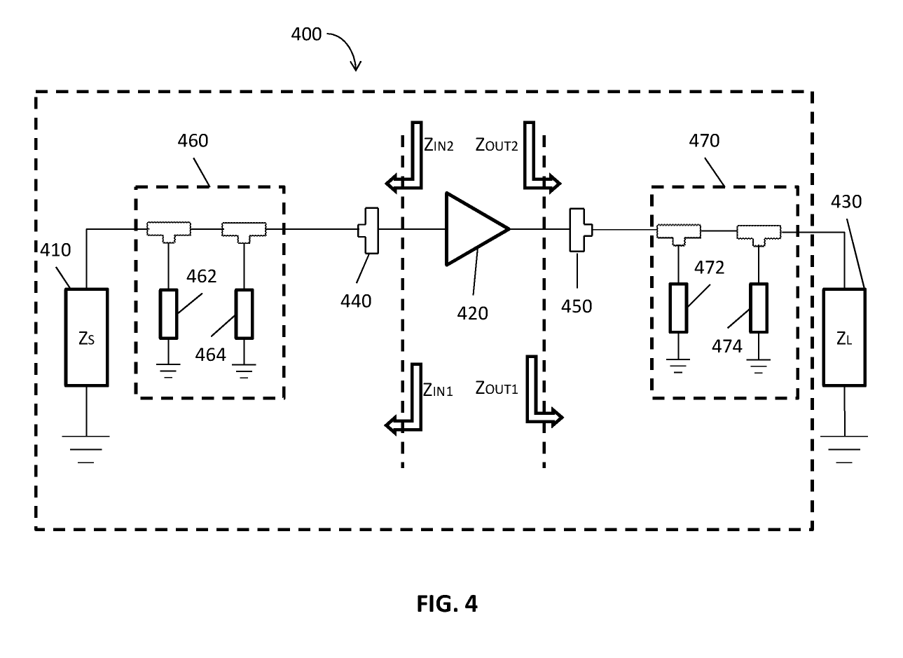

FIG. 4 shows a circuit diagram illustrating a dual band microwave generator 400, which can generate and concurrently amplify signals at two different and independent frequencies, f₁ and f₂. The dual band microwave generator 400 includes a microwave source 410, power amplifier 420, an input impedance matching circuit 440, an output impedance matching circuit 450, an auxiliary input impedance matching circuit 460, and an auxiliary output impedance matching circuit 470. The input and output matching circuits 440 and 450 are for matching impedance at the first frequency f₁, while the auxiliary input and output impedance matching circuits 460 and 470 are for matching impedance at the second frequency f₂.

The microwave source 410 is shown as a source impedance Z_{S} and the energy delivery device 430 is connected to the output port of the dual band microwave generator 400 and is shown as a load impedance Z_{L}. As described above in FIG. 3, the input and output impedance matching circuits 440 and 450 match impedance at the first frequency f₁, as the input and output impedance matching circuits 320 and 340 match impedance. The input impedance matching circuit 440 matches the source impedance Z_{S} to the input impedance Z_{IN1} of the power amplifier 420 at the first frequency f₁ and the output impedance matching circuit 450 matches the load impedance Z_{L} to the output impedance Z_{OUT1} of the power amplifier 420 at the first frequency f₁.

When the microwave source 410 generates a signal having the second frequency f₂ which is independent and substantially different from the first frequency f₁, the input and output impedance matching circuits 440 and 450 may not be able to match impedance values at the second frequency f₂. To compensate for this shortcoming, auxiliary input impedance matching circuit 460 matches the source impedance Z_{S} to the input impedance Z_{IN2} of the power amplifier 420 at the second frequency f₂, and the auxiliary output impedance matching circuit 470 match the load impedance Z_{L} to the output impedance Z_{OUT2} of the power amplifier 420 at the second frequency f₂.

The auxiliary input impedance matching circuit 460 includes a first shunt stub 462 and a second shunt stub 464, which are located between the input impedance matching circuit 440 and the source 410 in parallel. The first and second shunt stubs 462 and 464 have a 90 degree electrical length of the first frequency f₁, or a quarter wavelength of the first frequency f₁, meaning that the physical length S_{L} of the first and the second shunt stubs 462 and 464 is substantially equal to one quarter of the wavelength of the first frequency f₁ shown below:

$${S}_{L} = \frac{1}{4} λ = \frac{1}{4} \cdot \frac{v}{{f}_{1}} ,$$

where λ is the wavelength of the frequency f₁, and ν represents the speed of light or, in this situation, ν represents a speed of electrons moving in the first and second shunt stubs 462 and 464.

Generally, the speed of electrons moving in a transmission line is less than the speed of light due to the characteristic impedance. Thus, when considering the characteristic impedance, the quarter wavelength of the transmission line is shown below:

$${S}_{L} = \frac{1}{4} λ = \frac{1}{4} \cdot \frac{{v}_{c}}{\sqrt{{ɛ}_{r}}} \frac{1}{{f}_{1}} ,$$

where ν_{c} is the speed of light and ∈ᵣ is the dielectric constant, which is related to the characteristic impedance of the transmission line. The quarter wavelength shunt stub of a frequency f₁ is considered as open circuit resulting infinite impedance or simply does not affect the impedance of the whole circuit at the frequency f₁.

In practice, the physical length of a shunt stub may not be exactly equal to the quarter wavelength of a frequency due to other related parameters in transmission lines. Thus, the physical length of a shunt stub may be approximated so that the physical length of the shunt stub may be greater or less than the exact quarter wavelength of the frequency and may also be tuned or adjusted.

Since the first and second shunt stubs 462 and 464 of the auxiliary input impedance matching circuit 460 have the quarter wavelength of the first frequency f₁, the impedance values of the first and second shunt stubs 462 and 464 at the first frequency f₁ are infinity, ∞. Thus, when the input matching circuit 440 sees the impedance of the auxiliary input matching circuit 460 and the source Z_{S}, the input impedance matching circuit 440 only sees the source Z_{S}, as shown in the following calculation:

$$\frac{1}{\frac{1}{{Z}_{s}} + \frac{1}{∞} + \frac{1}{∞}} = \frac{1}{\frac{1}{{Z}_{s}} + 0 + 0} = {Z}_{s} .$$

In this way, the auxiliary input impedance matching circuit 460 does not affect the source impedance Z_{S} at the first frequency f₁. However, the impedance of the input impedance matching circuit 440 changes at the second frequency f₂. Thus, the input impedance matching circuit 440 and the auxiliary input impedance matching circuit 460 together can match the source impedance Z_{S} to the input impedance Z_{IN1} at the first frequency f₁ and to Z_{IN2} at the second frequency f₂ of the power amplifier 420, where f₁ and f₂ are different and independent from each other. The auxiliary output impedance matching circuit 470 is similar to the auxiliary input impedance matching circuit 460 and includes a third shunt stub 472 and a fourth shunt stub 474, which are located between and are in parallel with the output impedance matching circuit 450 and the load Z_{L}. The third and fourth shunt stubs 472 and 474 have a quarter wavelength of the first frequency f₁, meaning that the length S_{L} of the third and fourth shunt stubs 472 and 474 is also substantially equal to one quarter of the wavelength of the first frequency f₁. As with quarter wave length stubs 462 and 464, the quarter wavelength shunt stubs 472, 474 are considered open circuit resulting infinite impedance and simply do not affect the output impedance of the whole circuit at the second frequency f₂, meaning that the output impedance matching circuit 450 only sees the load Z_{L}, as described above with respect to input impedance matching circuit 440.

The output impedance matching circuit 450 and the auxiliary output impedance matching circuit 470 work the same way as the input impedance matching circuit 440 and the auxiliary input impedance matching circuit 460. Specifically, the output impedance matching circuit 450 matches the load impedance Z_{L} to the output impedance Z_{OUT1} of the power amplifier 420 at the first frequency f₁. And the auxiliary output impedance matching circuit 470 matches the load impedance Z_{L} to the output impedance Z_{OUT2} of the power amplifier 420 at the second frequency f₂, while the auxiliary output impedance matching circuit 470 does not affect the load impedance Z_{L} at the first frequency f₁.

By electrically connecting these auxiliary input and output impedance matching circuits 460 and 470 around the power amplifier 300 of FIG. 3, the dual band microwave generator 400 can perform signal generation and concurrent amplification of signal at two independent and different frequencies. In this way, the size and cost of making a dual band microwave generator 400 may reduce.

The parameters of the shunt stubs 462, 464, 472, and 474 may be variable such that they can be tuned to allow f₂ to be selected from a number of frequencies, without departing from the scope of the present disclosure.

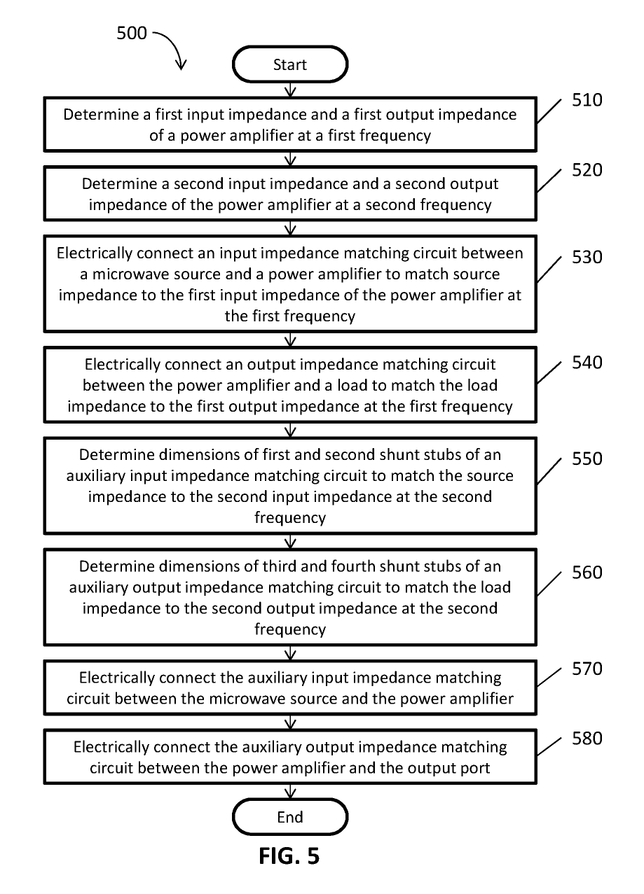

FIG. 5 shows a flowchart illustrating the method 500 for designing a dual band power amplifier circuitry of a dual band microwave generator to concurrently match impedance at two independent and different frequencies.

The method starts at step 510, in which a first input impedance and a first output impedance of a power amplifier of the dual band power amplifier circuitry are determined for predetermined performance of the dual band power amplifier circuitry. The first input impedance may be an impedance value, seen from the microwave source of the dual band microwave generator, of the power amplifier of the dual band power amplifier circuitry at a first frequency and the first output impedances may be an impedance value, seen from an output port of the dual band power amplifier circuitry, of the power amplifier at the first frequency. In step 520, a second input impedance and a second output impedance of the power amplifier are determined for predetermined performance of the dual band power amplifier circuitry. The second input impedance may be the impedance value, seen from the microwave source, of the power amplifier at a second frequency and the second output impedances may be an impedance value, seen from the output port of the dual band power amplifier circuitry, of the power amplifier at the second frequency.

In step 530, an input impedance matching circuit is electrically connected between the microwave source and the power amplifier to match the source impedance to the first input impedance of the power amplifier at the first frequency. In step 540, an output impedance matching circuit is also electrically connected between the output port of the dual band power amplifier circuitry and the power amplifier to match the load impedance to the first output impedance of the power amplifier at the first frequency.

As to matching impedance, at the first frequency, an imaginary part of the source impedance and an imaginary part of the first input impedance of the power amplifier have a substantially equal magnitude and have 180 degree out of phase meaning that they have different signs, and, at the first frequency, an imaginary part of the load impedance and an imaginary part of the first output impedance of the power amplifier have a substantially equal magnitude and have 180 degree out of phase. In the same way, at the second frequency, an imaginary part of the source impedance and an imaginary part of the second input impedance of the power amplifier have a substantially equal magnitude and have 180 degree out of phase, and, at the second frequency, an imaginary part of the load impedance and an imaginary part of the second output impedance of the power amplifier have a substantially equal magnitude and have 180 degree out of phase.

In step 550, dimensions of first and second shunt stubs of an auxiliary input impedance matching circuit are determined to match the source impedance to the second input impedance of the power amplifier at the second frequency. The first and second shunt stubs have substantially equal length, i.e., one quarter wavelength of the first frequency. Since the first and second shunt stubs act as an open circuit at the first frequency, the auxiliary input impedance matching circuit does not affect the source impedance at the first frequency. In step 560, dimensions of third and fourth shunt stubs of an auxiliary output impedance matching circuit are determined to match the load impedance to the second output impedance of the power amplifier at the second frequency. The third and fourth shunt stubs have a substantially equal length, i.e., one quarter wavelength of the first frequency. Since the third and fourth shunt stubs act as an open circuit at the first frequency, the auxiliary output impedance matching circuit does not affect the load impedance at the first frequency.

Dimension of shunt stub includes width, length, and thickness. The narrower the width or the length is, the larger the impedance is, and the thicker the thickness is, the larger the impedance is. Thus, by adjusting the width, length, or the thickness of the shunt stubs, the auxiliary input and output impedance matching circuits may be tuned to match the source and load impedances to the input and output impedance of the power amplifier at two different and independent frequencies. Here, the lengths of the first, second, third, and fourth shunt stubs are fixed to the quarter wavelength of the first frequency. Nevertheless, the lengths may also be adjusted to compromise real world defects in the shunt stubs to act as a quarter wavelength shunt stub.

In step 570, the auxiliary input impedance matching circuit is electrically connected between the microwave source and the power amplifier. The first and second shunt stubs of the auxiliary input impedance matching circuit are electrically connected in parallel with the microwave source and the power amplifier. In step 580, the auxiliary output impedance matching circuit is electrically connected between the power amplifier and the output load. The third and fourth shunt stubs of the auxiliary output impedance matching circuit are electrically connected in parallel with the power amplifier and the output port of the dual band power amplifier circuitry. By following these steps of the method 500, the dual band power amplifier circuitry can amplify signals generated by the microwave source to a predetermined magnitude concurrently at two independent and different frequencies.

In an embodiment, the auxiliary input impedance matching circuit, the input matching circuit, the power amplifier, the output impedance matching circuit, and the auxiliary output impedance matching circuits are electrically connected serially in order. In another embodiment, the auxiliary input impedance matching circuit and the input matching circuit may change their order of connection, and the output impedance matching circuit and the auxiliary output impedance matching circuits may also change their order of connection.

Since other modifications and changes may be made to fit particular operating requirements and environments, it is to be understood by one skilled in the art that the present disclosure is not limited to the examples described in the present disclosure and may cover various other changes and modifications which do not depart from the spirit or scope of this disclosure.

What is claimed is:

1. A dual band power amplifier comprising:
a power amplifier having an input and an output and configured to amplify input signals at a first frequency and a second frequency;
a first matching circuit electrically connected to the output of the power amplifier and configured to match a load impedance to an output impedance of the power amplifier at the first frequency;
a first auxiliary circuit electrically connected to the output of the power amplifier, the first auxiliary circuit having at least two shunt stubs, wherein the first matching circuit and the first auxiliary circuit are configured to match the load impedance to the output impedance of the power amplifier at the second frequency;
a second matching circuit electrically connected to the input of the power amplifier and configured to match a source impedance to an input impedance of the power amplifier at the first frequency; and
a second auxiliary circuit electrically connected to the input of the power amplifier, the second auxiliary circuit having at least two shunt stubs, wherein the second matching circuit and the second auxiliary circuit are configured to match the source impedance to the input impedance of the power amplifier at the second frequency,
wherein the at least two shunt stubs of the first auxiliary circuit and the at least two shunt stubs of the second auxiliary circuit have a length substantially equal to a quarter wavelength of the first frequency.

2. The dual band power amplifier according to claim 1, wherein the first frequency is substantially greater than the second frequency.

3. The dual band power amplifier according to claim 2, wherein the first frequency is 2450 megahertz (MHz) and the second frequency is 915 MHz.

4. The dual band power amplifier according to claim 1, wherein the first matching circuit, second matching circuit, first auxiliary circuit, second auxiliary circuit, and the power amplifier are electrically connected by a transmission line.

5. The dual band power amplifier according to claim 4, wherein a characteristic impedance of an input portion of the transmission line is equal to the source impedance.

6. The dual band power amplifier according to claim 4, wherein a characteristic impedance of an output portion of the transmission line is equal to the load impedance.

7. The dual band power amplifier according to claim 1, wherein, at the first frequency, an imaginary part of the source impedance and an imaginary part of the input impedance of the power amplifier have a substantially equal magnitude and are 180° out of phase, and, at the first frequency, an imaginary part of the load impedance and an imaginary part of the output impedance of the power amplifier have a substantially equal magnitude and are 180° out of phase.

8. The dual band power amplifier according to claim 1, wherein, at the second frequency, an imaginary part of the source impedance and an imaginary part of the input impedance of the power amplifier have a substantially equal magnitude and are 180° out of phase, and, at the second frequency, an imaginary part of the load impedance and an imaginary part of the output impedance of the power amplifier have a substantially equal magnitude and are 180° out of phase.

9. The dual band power amplifier according to claim 1, wherein the at least two shunt stubs of the first auxiliary circuit are electrically connected in parallel to the output of the power amplifier.

10. The dual band power amplifier according to claim 1, wherein the at least two shunt stubs of the second matching circuit are electrically connected in parallel to the input of the power amplifier.

11. A microwave generator comprising:
a source of microwave energy generating input signals at a first frequency and a second frequency and having a source impedance;
a power amplifier having an input and an output and configured to amplify the input signals received from the source at the first frequency and the second frequency;
a load electrically connected to the output of the power amplifier and having a load impedance;
a first matching circuit electrically connected to the output of the power amplifier and configured to match the load impedance to an output impedance of the power amplifier at the first frequency;
a first auxiliary circuit electrically connected to the output of the power amplifier, the first auxiliary circuit having at least two shunt stubs, wherein the first matching circuit and the first auxiliary circuit are configured to match the load impedance to the output impedance of the power amplifier at the second frequency;
a second matching circuit configured to match the source impedance to an input impedance of the power amplifier at the first frequency; and
a second auxiliary circuit electrically connected to the input of the power amplifier, the second auxiliary circuit having at least two shunt stubs, wherein the second matching circuit and the second auxiliary circuit are configured to match the source impedance to the input impedance of the power amplifier at the second frequency,
wherein the at least two shunt stubs of the first auxiliary circuit and the at least two shunt stubs of the second auxiliary circuit have a length substantially equal to a quarter wavelength of the first frequency.

12. The microwave generator according to claim 11, wherein the first frequency is substantially greater than the second frequency.

13. The microwave generator according to claim 12, wherein the first frequency is 2450 megahertz (MHz) and the second frequency is 915 MHz.

14. The microwave generator according to claim 11, wherein the source of microwave energy, first matching circuit, second matching circuit, first auxiliary circuit, second auxiliary circuit, and the power amplifier are electrically connected by a transmission line.

15. The microwave generator according to claim 14, wherein a characteristic impedance of an input portion of the transmission line is equal to the source impedance.

16. The microwave generator according to claim 14, wherein a characteristic impedance of an output portion of the transmission line is equal to the load impedance.

17. The microwave generator according to claim 11, wherein, at the first frequency, an imaginary part of the source impedance and an imaginary part of the input impedance of the power amplifier have a substantially equal magnitude and are 180° out of phase, and, at the first frequency, an imaginary part of the load impedance and an imaginary part of the output impedance of the power amplifier have a substantially equal magnitude and are 180° out of phase.

18. The microwave generator according to claim 11, wherein, at the second frequency, an imaginary part of the source impedance and an imaginary part of the input impedance of the power amplifier have a substantially equal magnitude and are 180° out of phase, and, at the second frequency, an imaginary part of the load impedance and an imaginary part of the output impedance of the power amplifier have a substantially equal magnitude and are 180° out of phase.

19. The microwave generator according to claim 11, wherein the at least two shunt stubs of the first auxiliary circuit are electrically connected in parallel to the output of the power amplifier.

20. The microwave generator according to claim 11, wherein the at least two shunt stubs of the second matching circuit are electrically connected in parallel to the input of the power amplifier.
